(19) United States
(12) Patent Application Publication
(10) Pub. No.: US 2005/0220666 A1
(43) Pub. Date: Oct. 6, 2005

(54) System and method for sterilization of personal and/or household items

(76) Inventor: Terry L. Foster, The Woodlands, TX (US)

Correspondence Address: Terry L. Foster, 83 West Ardsley Square PI., The Woodlands, TX, 77382, US

(21) Appl. No.: 11/088,235

(22) Filed: Mar. 23, 2005

Related U.S. Application Data
(60) Provisional application No. 60/555,734, filed on Mar. 23, 2004.

(51) Int. Cl. A61L 2/20

(52) U.S. Cl. 422/28; 422/292; 422/37; 422/34

(57) ABSTRACT

The present invention provides a system and methods for vapor phase sterilization of mail or other items to be delivered, including sterilization during transit of the parcels. In addition the invention provides compositions and methods for sterilization of personal or domestic items.

The present application claims the benefit of U.S. Provisional Application Ser. No. 60/555,734, filed Mar. 23, 2004, which is expressly incorporated herein by reference.

### FIELD OF THE INVENTION

The present invention relates generally to sterilization and more particularly to methods and compositions for sterilization of personal or household items, including mail, by vapor phase reagents.

### BACKGROUND OF THE INVENTION

Various methods of sterilization have been used in the past for the sterilization of different types of articles including disposable and reusable medical equipment, foods and food containers. Sterilization by steam or by dry heat has been extensively used in the past. Sterilization by heat, either wet or dry, is not useful to sterilize materials that are adversely affected by such heat or steam. Ethylene oxide gas has also been used but suffers from the drawback that it may leave toxic residues on the articles to be sterilized. The extended aeration cycles required to remove residual ethylene oxide from some sterilized items also makes ethylene oxide sterilization excessively long.

The use of plasma to sterilize containers was suggested in U.S. Pat. No. 3,383,163. Plasma is an ionized body of gas which may be generated by the application of power from different sources. The ionized gas will contact microorganisms on the surfaces of the items to be sterilized and effectively destroy the microorganisms.

Prior plasma sterilization systems have not been put into wide commercial use because of the limitations on the time required to effect sterilization, the temperature obtained in the sterilization process or the particular requirements of some of the processes that would require post-sterilization packaging.

Hydrogen peroxide has been known to have bactericidal properties and has been used in solutions to kill bacteria on various surfaces. U.S. Pat. No. 4,437,567 discloses the use of aqueous hydrogen peroxide solutions at low concentrations, i.e., 0.01% to 0.10% by weight, to sterilize packaged products for medical or surgical use. In addition, hydrogen peroxide has been used in large scale vapor phase sterilization of buildings.

Despite these advances in sterilization technologies and their use in commercial, large scale settings, these methods have not been adapted to or made their way into the general population. Reasons for this include the use, in some instances, of sophisticated devices, the expense of dedicated, commercial sterilization equipment, the use of potentially harmful chemicals, and the use of machines that use extreme conditions and therefore are not suitable for household use.

The need for small scale or household sterilization methods and devices has only been compounded by fears of and the reality surrounding recent terror threats. Today more than ever the public is faced with the reality that biological attacks pose a significant threat to society. This is no more clearly exemplified than by examining the recent biological attacks of anthrax that was sent through the mail.

Previously there has not existed a need for methods for sterilization of items to be delivered, such as mail, parcels, or packages. However, the recent bioterrorism events have highlighted the extreme urgency of new and improved methods and compositions for sterilization of public items such as mail as it is dispatched and handled in mass transit.

An additional threat faced by the public is the increased incidence of drug resistant or drug tolerant strains of microorganisms. While advances in pharmaceuticals attempt to control infection by these microorganisms, an additional attack could be waged against them with more efficient and widespread sterilization. However, there is currently not a method or device available to the public to successfully kill these organisms or sterilize items infected with or that have even been in contact with such organisms.

Accordingly, there exists a significant need for new methods and devices to be used and found in the general public and/or in households for sterilization.

### SUMMARY OF THE INVENTION

Accordingly, the present invention provides a system for sterilization of personal and household items as desired. The system includes a chamber that has sealable opening, automated or manual controls and a sterilization system. The sterilization system includes a container that contains a sterilization reagent, a dispenser connecting the container to the interior of the chamber, and a mechanism for evacuating said chamber.

In addition the invention provides a method of sterilization with safe materials and methods which terminate with environmentally acceptable by-products providing consumer-usable methods and materials.

In addition, the invention provides a sterilization apparatus comprising a collapsible sterilization housing, wherein said housing is sealable and a sterilant container configured to disperse a sterilant into said housing. In some embodiments, the collapsible sterilization unit contains braces to provide a rigid support. However, in alternate embodiments the sterilization unit does not contain supporting braces.

Additionally, the invention provides personal or domestic appliances, such as a dishwasher adapted for cycles of non-detergent sterilization

### BRIEF DESCRIPTION OF THE DRAWINGS

FIG. 1 depicts a sterilization system of the present invention including a housing 1, a sterilant container 3, a connector connecting the container to the housing 2, and optionally a device for removing the sterilant end-product 4 and optionally a waste collection receptacle 5.

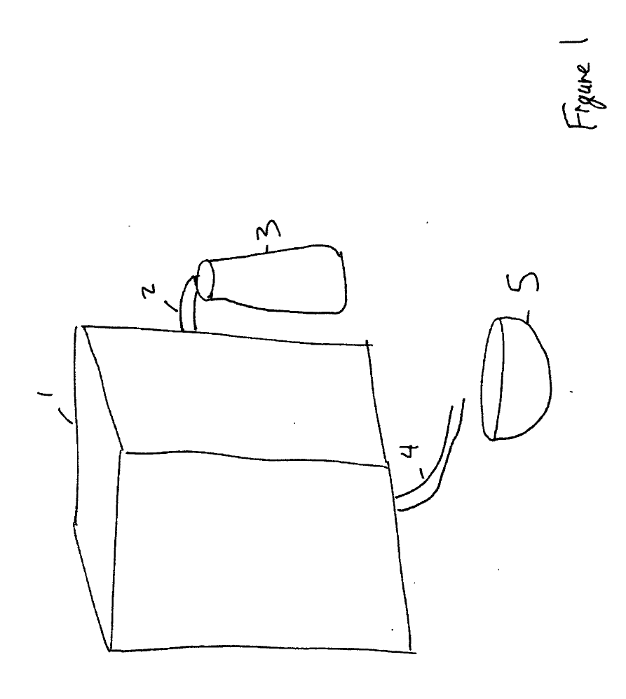

FIG. 2 depicts a sterilization system of the present invention including a housing 1 and a sterilant container 3 connected directly to the housing.

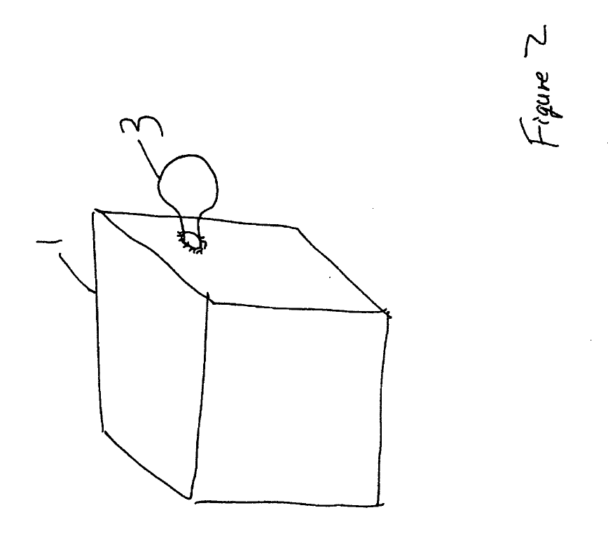

FIG. 3 depicts a collapsible sterilization according to the invention. A collapsible housing 7 is held upright by braces 6. The housing is of a pliable, flexible or collapsible material as indicated by the shading.

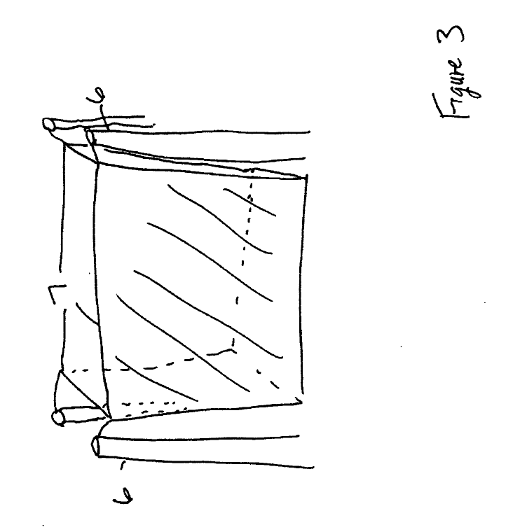

### DETAILED DESCRIPTION OF THE INVENTION

Methods for sterilization of various items are known. However, new technologies have not been applied to home or personal use. Currently there are not methods for sterilization of personal or household items based upon technologies developed in recent years and proven in commercial applications. Generally, personal and household sterilization is still performed as it has been for years. Given that bioterrorism has introduced new threats of microbial risks into society, there is an unmet need for new methods of personal and household item sterilization. By sterilization of personal or household items is meant sterilization of items in the general population as opposed to sterilization of items in a specialized field or location, such as a laboratory. Accordingly, the present invention provides new systems to combat the higher, more threatening contamination risks of today. As such, this not only provides application of new, effective sterilization technologies for personal and household use; it works in concert with homeland security to place control of personal threat into personal hands.

The invention provides compositions and methods for sterilization of personal and household items. While various methods for household sterilization are known, i.e. steam, dry heat, liquid chemicals such as bleach, and the like, vapor phase, i.e. gaseous phase sterilization finds particular use as applied to household and personal use. That is because the improved technology is effective against microbial vegetative cells and spores and viruses, yet it does not damage the items. It decontaminates the exterior and, in appropriate cases, the interior of items. It does not leave a toxic residue; it can be applied in a manual or automated format; it is cost-effective; and the by-products of the reaction are easily disposable with no threat to the environment. Accordingly the invention provides a method of vapor-phase sterilization of personal and household items in domestic arenas.

In addition, the invention provides compositions and methods for sterilization of mail or other parcels in transit.

The apparatus of the invention includes a housing for insertion of the item or items to be sterilized. As such, the apparatus includes at least one opening for insertion and removal of the item to be sterilized, but it also may have more than one opening. In addition the apparatus includes a sterilization container with an inlet connected to the housing to allow introduction of sterilizing reagent and removal of final by-products of sterilization, or the apparatus may include an inlet for the addition of or introduction of a sterilization reagent and an outlet for the removal or evacuation of the final by-product or end-product. The outlet may be connected to a waste receptacle or, when the end-product is water or other useful by-product, the outlet may be connected to a device for recycling the by-product In the case where water is the by-product, the water may be recycled, for example, by using it for irrigation etc..

Alternatively, the apparatus is self contained in that the sterilization agent is placed directly into the housing. When the process is complete, the end-products are removed from the housing. By “end-products” or “by-products” and grammatical equivalents herein is meant the product resulting from the break-down of the sterilization reagent following the sterilization process. The housing may include a pan or other collecting container for holding the sterilization agent and/or by-product, e.g. water (when hydrogen peroxide is the agent). In addition, as described herein, the housing also may include a desiccant that absorbs the end-product. Preferred desiccants include, but are not limited to conventional solid desiccants such as silica gel, activated alumina, lithium chloride salt, and molecular sieves, titanium silicate, a class of material known in the art as “1 m,”. Liquid desiccants include, but are not limited to, lithium chloride, lithium bromide, calcium chloride, and triethylene glycol solutions.

In addition, the apparatus includes appropriate valves and monitoring devices to allow proper introduction, holding-time, and removal of sterilizing reagent in a manual or automated format. An embodiment of the application is to utilize an appropriate sealable container, exemplified by, but not limited to, a household pressure cooker fitted at its inlet with a tubing and “T” valve. By “sealable”, “sealed” or grammatical equivalents herein is meant able to be made airtight. Such seals can be made by using clamps, o-rings, pressure, e.g. internal evacuating pressure and/or external application of pressure to close a sealable opening, adhesives, zippers and gaskets, VELCRO®, combinations of these, and the like. However, it is appreciated that while the apparatus may is sealable, sterilant and waste products are still able to be delivered to and removed from the apparatus, respectively. That is, the system is configured as a closed system including sterilant and sterilant delivery components, the container containing items to be sterilized and in some embodiments a waste removal component. By “closed system” is meant that the components of the system are able to be closed off such that the system is self contained and external air is not allowed in the system.

Material to be sterilized is placed into the apparatus, e.g. pressure cooker-type device, which is then sealed. In one preferred use, the chamber can be evacuated by appropriate aspiration In another preferred use, the sterilant gaseous source is provided in a pressurize canister. The canister is connected, either directly, e.g. direct attachment to the apparatus, or indirectly, e.g. by the use of a connector, such as a tube, to the container to allow introduction of sterilant to the container or chamber. Evacuation of the chamber or supply of pressurized canister of sterilant assures distribution of sterilant to all parts of the interior of the chamber. In this example, the device, e.g., pressure cooker-type device, houses an elevated plate on its interior bottom. Addition of appropriate desiccant assures removal of waste product (water in the case of hydrogen peroxide) upon completion of sterilization

Accordingly, in one embodiment the invention provides a housing with appropriate controls and monitoring devices with requisite features of the sterilization apparatus described above, such as inlet ports for distribution of vapor phase reagents and outlet ports for removal of by-products of the sterilization reaction. In this embodiment the housing also includes an opening for the insertion of the item to be sterilized. However, there is also a sealing mechanism that closes to allow for sterilization in the closed container. There may also be a pressure regulator on the device, a sterilant concentration monitor on the device, and/or a heater on the device as is determined to be necessary.

In addition, the apparatus of the invention may include a sensor for detecting centration and/or presence of vapor phase sterilants as disclosed in U.S. Patent application publication 20050019206, 20040265170 and 20050013726, which are expressly incorporated herein by reference.

Methods and other devices utilizing vapor-phase sterilization are disclosed in U.S. Pat. Nos. 5,007,232, 4,956,145, 4,643,876, 5,876,666, 4,169,123, 5,445,792, 5,508,009, 4,863,688, and 5,552,320, all of which are expressly incorporated herein by reference. A mist sterilization device is disclosed in U.S. patent application publication 2005/0042130, which is expressly incorporated herein by reference.

By “sterilizing reagent” or “sterilant” or equivalent herein is meant an agent which effectively kills spores, vegetative cells, and viruses without adversely affecting the item being sterilized. The agent preferably is a non-detergent based sterilizing agent. In a preferred embodiment the sterilization agent includes agents such as hydrogen peroxide or chlorine dioxide. Other agents or reagents that can be used with the invention include ethylene oxide, peracetic acids, other peroxides, and ozone. When items are to be sterilized, hydrogen peroxide is particularly preferred, although chlorine dioxide also finds uses in the invention.

Hydrogen peroxide is preferred because it can be used at a variety of concentrations, but is effective even in low concentrations. It can be used with low humidity and with ambient temperatures, although a variety of temperatures and humidities can be used. Importantly, when hydrogen peroxide is used, the by-products of the reaction are water and oxygen, both of which can be readily removed from the reaction chamber and released or trapped for disposal. In addition, these by-products do not harm the items being sterilized. Finally, this sterilization agent is highly germicidal and sporocidal, is readily available and is cost-effective.

Chlorine dioxide also is highly germicidal and sporocidal, but it generally requires higher humidity, and the by-products are more difficult to manage. That is, while a variety of materials may be used as vapor phase sterilant in this process, the preferred embodiment includes utilization of hydrogen peroxide or chlorine dioxide. Hydrogen peroxide is preferred.

The hydrogen peroxide can be provided as a solution that is flash vaporized to provide vapor phase hydrogen peroxide for the sterilization process. This new technology is described in more detail in U.S. Pat. Nos. 5,007,232, 4,956,145, 4,643,876, and 5,508,009. The hydrogen peroxide can be provided as a dry reagent that can be activated to release vapor phase hydrogen peroxide for the sterilization process. This new technology is described in more detail in U.S. Pat. No. 5,876,666. All references are expressly incorporated herein by reference.

chlorine dioxide provides preferred utility in an embodiment of this invention, it can be provided as a result of reacting a sodium chlorite solution with an organic acid as described in several patents and references including U.S. Pat. Nos. 6,042,802, 5,326,646, and 5,290,524. Additionally, it can be provided from dry reagents as described in U.S. Pat. No. 5,639,295. All references are expressly incorporated herein by reference.

Accordingly, the invention provides an apparatus or device for vapor phase sterilization. In one embodiment the apparatus is a mail truck, airplane, or housing into which a mail truck or airplane is placed. Alternatively the apparatus could be a domestic apparatus such as a personal sterilizing device. In another embodiment the apparatus is a mobile sterilization facility. In this embodiment the sterilization facility includes the sterilization, reagent, inlet and in some embodiments the outlet as described previously. In addition, the inventive facility is a portable facility, such as a tent. In one embodiment a conventional tent is used. However, in a preferred embodiment an inflatable tent is used.

Inflatable tents offer several advantages over conventional tents in that they do not require the hardware that conventional tents require. Inflatable tents are assembled in a fraction of the time with a fraction of the effort of conventional tents. Inflatable tents are described in more detail in U.S. Pat. U.S. Pat. Nos. 6,810,896, 6,722,084, 6,708,451, 6,598,613, 6,508,850, 6,263,617, 6,167,898, 5,987,822, 5,964,222, all of which are expressly incorporated herein by reference. In a preferred embodiment, as with other embodiments, the inflatable tent is sealable as described above, e.g. able to be made air tight such that it is a closed system. Preferably the tent will have controlled air flow into the tent with appropriate filters, such as HEPA filters to ensure cleanliness of the interior of the tent.

In another embodiment of the invention the home sterilization apparatus is a household appliance similar to the size and configuration of routine microwave ovens, although not limited to this size. In one embodiment the apparatus is from about 6×6×6 inches to about 10×10×10 feet; in a more preferred embodiment the apparatus is from about 6×6×6 inches to about 6×6×6 feet; in a more preferred embodiment the apparatus is from about 6×6×6 inches to about 3×3×3 feet and most preferably is from about 6×6×6 inches to about 2×2×2 feet or 1×1×1×feet.

The apparatus can contain a microprocessor controller and a touch-pad control panel to provide control of the sterilization process. In this embodiment, an item or items are placed into the interior chamber of the apparatus, and the door is sealed. Specifications for the sterilization cycle are input into the control panel, the control panel is activated, and the microprocessor controls the sterilization and evacuation cycle. The microprocessor can be programmed to control and/or monitor any one, a combination thereof or all of the following: time, humidity, sterilant concentration, temperature, airflow (if required), and other required variables. Algorithms programmed into the microprocessor assure sterilant contact with the items to be sterilized for the desired time, humidity, sterilant concentration, temperature, and other variables. The apparatus can be operated manually and may possess appropriate monitoring gauges and devices and control valves for introduction and removal of sterilant as necessary.

In a particularly preferred embodiment the sterilization device is a portable sterilization device. In this embodiment the device is configured such that it is able to be readily moved from place to place. In a preferred embodiment, the device is configured to be assembled and disassembled quickly. In this embodiment the components of the sterilization system, e.g. the housing, and sterilant container are separated and can be transported independently. The sterilization system can be readily re-assembled by re-connecting the components using screw connectors, snap connectors, zippers, VELCRO® hook and ring closures, or other connectors as are known in the art.

In a preferred embodiment the sterilization system housing is a collapsible housing. In this embodiment the walls of the sterilization system housing are not constantly rigid, but rather they are pliable. This is in contrast to the housings of the prior art. Thus, in some embodiments, the collapsible sterilization system does not contain rigid supports, such as braces and the like.

This embodiment finds particular use when combined with the portable sterilization device because the sterilization system can be disassembled, collapsed and stored or transported in a space considerably smaller than that of a conventional sterilizer.

Pliable and foldable materials that can be used in the invention include any non-porous, pliable materials. In some embodiments, the apparatus includes multiple layers that may serve unique purposes, such as a plastic/polymer interior, a canvas exterior, and in some embodiments an insulating layer. Preferred materials include plastics and polymers that include, but are not limited to polystyrene and copolymers of styrene and other materials, polypropylene, polyethylene, polyvinyl chloride (pvc), polybutylene, polyurethanes. Materials can include non porous, pliable material known to the art in the sports industry, infant/baby industry, and in the military, namely canvas covered, non porous, pliable material.

In one embodiment when it is desirous for the portable sterilization system to have rigid walls the housing may be supported by braces. By a “brace” or “braces” or grammatical equivalents is meant a support to provide rigidity to an otherwise flexible or pliable housing. In this embodiment the braces may be attached to the top and bottom of the housing They may be attached on the inside or outside of the housing. Alternatively, the housing may be placed on a frame that provides a brace. In this embodiment the frame is preferably metal or plastic and preferably is collapsible or foldable such that it too can be used with the portable sterilizer. In a preferred embodiment the brace(s) are inflatable support members. By inflatable support members is meant a device that is inflatable and provides support for the portable sterilization system. The inflatable support member may be made of material as described previously and may be constructed as described previously when describing inflatable tents.

Accordingly, in a preferred embodiment the present invention provides a portable sterilizer kit. Included in the kit are components to assemble the sterilizer as described herein. Preferred components include sterilant container, and sterilizer housing. Preferably the sterilizer housing is collapsible sterilizer housing. In addition the kit may contain connectors to connect the sterilant container to the housing. Alternatively, the sterilant container connects directly to a port on the housing. In some embodiments the kit also contains a brace for supporting the collapsible housing. Also included in the kit is an air pump, either manual pump or motored, e.g. electric, gas or battery powered); a vacuum supply also may be included to facilitate the evacuation of any inflatable devices.

In one embodiment the device is a conventional dish washer configured to sterilize the elements inside the apparatus. As is appreciated by those of skill in the art, conventional dishwasher includes liquid inlet system to supply water, liquid removal system, to remove water and waste, detergent supply component and requisite controls. However, the system of the present invention provides a dishwasher that is able to sterilize the contents of the device in the absence of a detergent. The dishwasher is configured to hold the sterilant or configured to have the sterilant input from a separate container. The configuration allows the dishwasher to be used in the conventional manner or used as a vapor-phase sterilization device for those items that cannot withstand dishwasher cycles. Thus, in some embodiments, the dishwasher includes liquid inlet system, liquid removal system, detergent receptacle, non-detergent sterilant container and requisite controls. In some embodiments the invention allows for conventional washing of dishes with detergents and sterilization with the non-detergent sterilant. In some embodiments, the invention allows for sterilization with non-detergent sterilants without the need for detergents.

In another embodiment of the invention the sterilization apparatus is the delivery vehicle, such as a parcel or mail delivery vehicle. By analogy to refrigerated delivery trucks, the delivery vehicle is equipped with sterilization features as described above. Refrigerated delivery trucks are described in more detail in U.S. Pat. Nos. 6,289,684, 5,514,345, 5,799,495, and 5,934,741, all of which are expressly incorporated herein by reference. Parcel delivery vehicles include but are not limited to delivery jeeps, trucks, vans, airplanes, and the like.

In addition the invention provides a method for sterilization of mail delivery vehicles. That is, the delivery vehicle as noted above are moved or placed in a sealable room and sterilized as described herein with vapor phase reagents. The sealable room may be a conventional room as in a building or may be in a collapsible or portable tent as described previously.

In yet another embodiment of the invention the sterilization occurs within the mail processing location or center. That is, while in the processing center, i.e. post office or even mail centers of mail recipients, the mail is sterilized. Sterilization may be accomplished by placing the items to be sterilized in a sealable enclosure, such as a room or container, into which is distributed the vapor phase sterilization reagent. In an alternative embodiment as the mail is routed through the post office, for example on a conveyor belt or analogous device, there is a particular station at which the mail stops, the sterilization station is sealed and sterilization reagents are introduced into the station. Following the procedure, the station is opened and the mail proceeds through processing.

In another embodiment, it is not only the mail that can be sterilized, but the mail processing equipment also can be sterilized by this method. Certain sterilization procedures, i.e. radiation sterilization, are limited to decontamination of the mail, but are not able to decontaminate the machinery involved in processing the mail. Thus, an important feature of the invention is the ability to seal an entire room and sterilize the machinery or other devices within the room.

Once made the invention provides a method for sterilization of mail or personal devices. As noted above, while vapor phase sterilization methods are known, it has not been appreciated how these methods could be applied to personal and household items including mail, parcels, toys, food utensils or implements to be sterilized at home or other non-commercial location.

In this embodiment, an item or items are placed into the interior chamber of the apparatus, and the door is closed or sealed. By use of appropriate valves, the desired centration or amount of sterilant will be introduced into the interior chamber and humidity be monitored and controlled simultaneously with temperature. Preferred concentrations for sterilant will be readily determined by one of skill in the art. When hydrogen peroxide is used, preferred ranges include from 0.1% to 100%, more preferably less than about 90% or 80% or 70% or 60% or 50% or 40% or 30% or 20% or 10% or 5% or 1%. Preferred concentrations include, but are not limited to about 0.46-0.87 mg/L with particularly preferred concentrations being >0.9 mg/L.

Sterilant will be allowed to contact items for the desired period of time. Additional sterilant can be added as defined by the process. At the termination of sterilization and by utilization of appropriate valves, sterilant by-products can be removed from the interior chamber be released directly to the environment or passed through appropriate trapping devices to contain by-products of the sterilization process.

The time for sterilization will vary depending on the item to be sterilized, the amount the item to be sterilized, temperature, humidity, sterilant concentration, and the size of the sing. However, determining optimum conditions for sterilization is well within the skill of one of ordinary skill in the art.

All references cited herein are incorporated by reference.

1. (canceled)

2. The device according to claim 8, wherein said sterilant container and said housing are connected by a tube.

3. The device according to claim 8, wherein said sterilant container is connected directly to said housing.

4. The device according to claim 8 wherein said sterilant container contains a sterilant.

5. (canceled)

6. (canceled)

7. (canceled)

8. A sterilizer device comprising:
a) a sealable housing;
b) a sterilant container configured to disperse said sterilant into said housing; and
c) microprocessors configured to control sterilization conditions,
wherein said device approximates the size of a conventional microwave oven.

9. (canceled)

10. (canceled)

11. (canceled)

12. (canceled)

13. (canceled)

14. (canceled)

15. (canceled)

16. (canceled)

17. (canceled)

18. The device according to claim 4, wherein said sterilant is selected from the group consisting of hydrogen peroxide, chlorine dioxide, ethylene oxide, peracetic acids, peroxides, and ozone.

19. The apparatus according to claim 4 further comprising support braces.
